(19) United States
(12) Patent Application Publication
(10) Pub. No.: US 2023/0131225 A1
(43) Pub. Date: Apr. 27, 2023

(54) Modulation of Microglia Activation

(71) Applicant: ABT Holding Company, Cleveland, OH (US)

(72) Inventors: Jason A. Hamilton, Shaker Heights, OH (US); Anthony E. Ting, Shaker Heights, OH (US); Robert W. Mayas, Shaker Heights, OH (US)

(21) Appl. No.: 17/956,347

(22) Filed: Sep. 29, 2022

Related U.S. Application Data
(63) Continuation of application No. 13/072,084, filed on Mar. 25, 2011, now abandoned.
Application No. 13/072,084 is a continuation-in-part of application No. PCT/US2011/025991, filed on Feb. 24, 2011.
(60) Provisional application No. 61/315,655, filed on Mar. 19, 2010.
(60) Provisional application No. 61/308,082, filed on Feb. 25, 2010.

(51) Int. Cl. G01N 33/50; A61K 35/28; C12Q 1/68; G01N 33/53

(52) U.S. Cl. CPC G01N 33/5058; A61K 35/28; G01N 33/5005; C12Q 1/68; G01N 33/53; G01N 33/5023; G01N 2500/10; G01N 2500/04

(57) ABSTRACT

The invention provides methods for treating pathological conditions associated with an undesirable inflammatory component. The invention is generally directed to reducing inflammation by administering cells that modulate microglia activation. The invention is also directed to drug discovery methods to screen for agents that modulate the ability of the cells to modulate microglia activation. The invention is also directed to cell banks that can be used to provide cells for administration to a subject, the banks comprising cells having desired levels of potency to modulate microglia activation.

### FIELD OF THE INVENTION

The invention provides methods for treating conditions associated with an undesirable inflammatory component, e.g., conditions of the central nervous system (CNS). The invention is generally directed to reducing inflammation by administering cells that modulate microglial activation. The invention is also directed to drug discovery methods to screen for agents that modulate the ability of the cells to modulate microglial activation. The invention is also directed to cell banks that can be used to provide cells for administration to a subject, the banks comprising cells having a desired potency for modulating microglial activation. The invention is also directed to compositions comprising cells of specific potency for modulating microglial activation, such as pharmaceutical compositions. The invention is also directed to methods for evaluating the dose efficacy of the cells in a patient by assessing the in vivo or in vitro activation of microglia. The invention is also directed to diagnostic methods conducted prior to administering the cells to a subject to be treated, including assays to assess the desired potency of the cells to be administered. The invention is further directed to post-treatment diagnostic assays to assess the effect of the cells on a subject being treated. The cells are non-embryonic stem, non-germ cells that can be characterized by one or more of the following: extended replication in culture and express markers of extended replication, such as telomerase, express markers of pluripotentiality, and have broad differentiation potential, without being transformed.

### SUMMARY OF THE INVENTION

The invention is broadly directed to methods for immunomodulation by modulating microglial activation.

The invention is more specifically directed to methods for immunomodulation within the CNS by modulating microglial activation.

The invention is also more specifically directed to methods for reducing microglial neurotoxic activation and/or increasing microglial neuroprotective activation in CNS conditions.

The invention is also more specifically directed to methods for reducing antigen presentation by microglia. Such reduction may result, for example, from reduced expression of one or more genes involved in the antigen presentation.

The invention is also more specifically directed to methods for modulating the activation state of microglia by means of secreted factors.

The invention is also more specifically directed to methods for modulating the activation state of microglia in CNS conditions by secreted factors that increase neuroprotective activation and/or reduce neurotoxic activation.

The invention is also directed to methods for driving microglia towards a TH2 (neuroprotective) immune response and/or away from a TH1 (neurotoxic) immune response in CNS injury.

The invention is also directed to methods for reducing CNS injury, including, but not limited to, ischemic stroke, multiple sclerosis, Alzheimer's Disease, ALS, Parkinson's Disease, hypoxic-ischemia, neonatal hypoxic ischemia, and traumatic brain or spinal cord injury, by modulating microglial activation.

The invention is also directed to methods for reducing CNS injury by increasing neuroprotective activation by microglia and/or reducing neurotoxic activation.

Factors that induce neuroprotective activation include, but are not limited to, CCL21 and CXCL10. Factors that suppress neurotoxic activation include, but are not limited to, TGFβ, CCL5, NGF, Galectin-1, Pentraxin-3, TGF-β, VEGF, BDNF, HGF, adrenomedullin, and thrombospondin.

Factors secreted by microglia during activation include, but are not limited to, oxygen radicals, NO, TNFα, Glu, quinolic acid, histamine, eicosanoids, NGF, BDNF, NT-4/5, TGFβ, GDNF, CNTF, IL-6, LIF, bFGF, HGF, PGn, and IL-3.

Factors secreted by microglia during neurotoxic activation include, but are not limited to, oxygen radicals, NO, TNFα, Glu, quinolic acid, histamine, and eicosanoids. Factors secreted by microglia during neuroprotective activation include, but are not limited to, NGF, BDNF, NT-4/5, TGFβ, GDNF, CNTF, IL-6, LIF, bFGF, HGF, PGn, and IL-3.

According to this invention, providing the above effects can be achieved by administering cells naturally (i.e., non-recombinantly) expressing and/or secreting one or more modulatory factors or medium conditioned by the cells. Cells include, but are not limited to, cells that are not embryonic stem cells and not germ cells, having some characteristics of embryonic stem cells, but being derived from non-embryonic tissue, and expressing and/or secreting one or more modulatory factors. The cells may naturally express/secrete one or more modulatory factors (i.e., not genetically or pharmaceutically modified to activate expression and/or secretion). However, natural expressors can be genetically or pharmaceutically modified to increase potency.

The cells may express pluripotency markers, such as oct4. They may also express markers associated with extended replicative capacity, such as telomerase. Other characteristics of pluripotency can include the ability to differentiate into cell types of more than one germ layer, such as two or three of ectodermal, endodermal, and mesodermal embryonic germ layers. Such cells may or may not be immortalized or transformed in culture. The cells may be highly expanded without being transformed and also maintain a normal karyotype. For example, in one embodiment, the non-embryonic stem, non-germ cells may have undergone at least 10-40 cell doublings in culture, such as 50, 60, or more, wherein the cells are not transformed and have a normal karyotype. The cells may differentiate into at least one cell type of each of two of the endodermal, ectodermal, and mesodermal embryonic lineages and may include differentiation into all three. Further, the cells may not be tumorigenic, such as not producing teratomas. If cells are transformed or tumorigenic, and it is desirable to use them for infusion, such cells may be disabled so they cannot form tumors in vivo, as by treatment that prevents cell proliferation into tumors. Such treatments are well known in the art.

Cells include, but are not limited to, the following numbered embodiments:

1. Isolated expanded non-embryonic stem, non-germ cells, the cells having undergone at least 10-40 cell doublings in culture, wherein the cells express oct4, are not transformed, and have a normal karyotype.

2. The non-embryonic stem, non-germ cells of 1 above that further express one or more of telomerase, rex-1, rox-1, or sox-2.

3. The non-embryonic stem, non-germ cells of 1 above that can differentiate into at least one cell type of at least two of the endodermal, ectodermal, and mesodermal embryonic lineages.

4. The non-embryonic stem, non-germ cells of 3 above that further express one or more of telomerase, rex-1, rox-1, or sox-2.

5. The non-embryonic stem, non-germ cells of 3 above that can differentiate into at least one cell type of each of the endodermal, ectodermal, and mesodermal embryonic lineages.

6. The non-embryonic stem, non-germ cells of 5 above that further express one or more of telomerase, rex-1, rox-1, or sox-2.

7. Isolated expanded non-embryonic stem, non-germ cells that are obtained by culture of non-embryonic, non-germ tissue, the cells having undergone at least 40 cell doublings in culture, wherein the cells are not transformed and have a normal karyotype.

8. The non-embryonic stem, non-germ cells of 7 above that express one or more of oct4, telomerase, rex-1, rox-1, or sox-2.

9. The non-embryonic stem, non-germ cells of 7 above that can differentiate into at least one cell type of at least two of the endodermal, ectodermal, and mesodermal embryonic lineages.

10. The non-embryonic stem, non-germ cells of 9 above that express one or more of oct4, telomerase, rex-1, rox-1, or sox-2.

11. The non-embryonic stem, non-germ cells of 9 above that can differentiate into at least one cell type of each of the endodermal, ectodermal, and mesodermal embryonic lineages.

12. The non-embryonic stem, non-germ cells of 11 above that express one or more of oct4, telomerase, rex-1, rox-1, or sox-2.

13. Isolated expanded non-embryonic stem, non-germ cells, the cells having undergone at least 10-40 cell doublings in culture, wherein the cells express telomerase, are not transformed, and have a normal karyotype.

14. The non-embryonic stem, non-germ cells of 13 above that further express one or more of oct4, rex-1, rox-1, or sox-2.

15. The non-embryonic stem, non-germ cells of 13 above that can differentiate into at least one cell type of at least two of the endodermal, ectodermal, and mesodermal embryonic lineages.

16. The non-embryonic stem, non-germ cells of 15 above that further express one or more of oct4, rex-1, rox-1, or sox-2.

17. The non-embryonic stem, non-germ cells of 15 above that can differentiate into at least one cell type of each of the endodermal, ectodermal, and mesodermal embryonic lineages.

18. The non-embryonic stem, non-germ cells of 17 above that further express one or more of oct4, rex-1, rox-1, or sox-2.

19. Isolated expanded non-embryonic stem, non-germ cells that can differentiate into at least one cell type of at least two of the endodermal, ectodermal, and mesodermal embryonic lineages, said cells having undergone at least 10-40 cell doublings in culture.

20. The non-embryonic stem, non-germ cells of 19 above that express one or more of oct4, telomerase, rex-1, rox-1, or sox-2.

21. The non-embryonic stem, non-germ cells of 19 above that can differentiate into at least one cell type of each of the endodermal, ectodermal, and mesodermal embryonic lineages.

22. The non-embryonic stem, non-germ cells of 21 above that express one or more of oct4, telomerase, rex-1, rox-1, or sox-2.

In one embodiment, the subject is human.

In view of the property of the cells to achieve the above effects, the cells can be used in drug discovery methods to screen for an agent that affects the ability of the cells to modulate microglia activation so as to be able achieve any of the above effects. Such agents include, but are not limited to, small organic molecules, antisense nucleic acids, siRNA, DNA aptamers, peptides, antibodies, non-antibody proteins, cytokines, chemokines, and chemo-attractants.

Because the effects described in this application can be caused by secreted factors, not only the cells, but also conditioned medium (and extracts thereof) produced from culturing the cells, is useful to achieve the effects. Such medium would contain the secreted factor(s) and, therefore, could be used instead of the cells or added to the cells. So, where cells can be used, it should be understood that conditioned medium (and extracts thereof) would also be effective and could be substituted or added.

In view of the property of the cells to achieve the above effects, cell banks can be established containing cells that are selected for having a desired potency to modulate the activation of microglia so as to be able to achieve any of the above effects. Accordingly, the invention encompasses assaying cells for the ability to modulate microglia activation and banking the cells having a desired potency. The bank can provide a source for making a pharmaceutical composition to administer to a subject. Cells can be used directly from the bank or expanded prior to use. Especially in the case that the cells are subjected to further expansion, after expansion it is desirable to validate that the cells still have the desired potency. Banks allow “off the shelf” use of cells that are allogeneic to the subject.

Accordingly, the invention also is directed to diagnostic procedures conducted prior to administering the cells to a subject. The pre-diagnostic procedures include assessing the potency of the cells to modulate microglia activation so as to be able to achieve one or more of the above effects. The cells may be taken from a cell bank and used directly or expanded prior to administration. In either case, the cells could be assessed for the desired potency. Especially in the case that the cells are subjected to further expansion, after expansion it is desirable to validate that the cells still have the desired potency. Or the cells can be derived from the subject and expanded prior to administration back to the subject (autologous). In this case, as well, the cells could be assessed for the desired potency prior to administration.

Although the cells selected for modulation are necessarily assayed during the selection procedure, it may be preferable and prudent to again assay the cells prior to administration to a subject for treatment to confirm that the cells still modulate microglia activation at desired levels. This is particularly preferable where the cells have been expanded or have been stored for any length of time, such as in a cell bank, where cells are most likely frozen during storage.

With respect to methods of treatment with cells that modulate microglia activation, between the original isolation of the cells and the administration to a subject, there may be multiple (i.e., sequential) assays for modulation. This is to confirm that the cells can still modulate microglia activation, at desired levels, after manipulations that occur within this time frame. For example, an assay may be performed after each expansion of the cells. If cells are stored in a cell bank, they may be assayed after being released from storage. If they are frozen, they may be assayed after thawing. If the cells from a cell bank are expanded, they may be assayed after expansion. Preferably, a portion of the final cell product (i.e., the cell preparation that is physically administered to the subject) may be assayed.

The invention further includes post-treatment diagnostic assays, following administration of the cells, to assess efficacy. The diagnostic assays include, but are not limited to, assays for factors expressed and/or secreted by activated microglia. Factors expressed in the neurotoxic activation state include, but are not limited to, TNF-α, IL-6, and matrix metalloproteases (MMPs). Factors expressed and/or secreted in the neuroprotective activation state may also be assayed.

The invention is also directed to a method for establishing the dosage of the cells by assessing the potency of the cells to modulate microglia activation so as to be able to achieve one or more of the above effects. In this case, the potency would be determined and the dosage adjusted accordingly.

Potency can be assessed by measuring the degree of microglia activation in vivo or in vitro by means of microglia gene expression or by the effects of microglia activation.

One may monitor microglia function (e.g., activation and presentation) to establish and maintain a proper dosage regimen. One could monitor the function at various levels, such as in vivo by means of circulating secreted factors expressed by activated microglia. One might also assay microglia that are derived from the patient in in vitro assays of gene expression or microglia function. Thus, the invention is directed to evaluating the dosage efficacy of the cells as an immunomodulator in the CNS in a patient by assessing and/or monitoring the in vivo activation of microglia.

The invention is also directed to compositions comprising a population of the cells having a desired potency, and, particularly, desired levels of modulating microglia activation. Such populations may be found as pharmaceutical compositions suitable for administration to a subject and/or in cell banks from which cells can be used directly for administration to a subject or expanded prior to administration. In one embodiment, the cells have enhanced (increased) potency compared to the previous (parent) cell population. Parent cells are as defined herein. Enhancement can be by selection of natural expressors or by external factors acting on the cells.

The methods and compositions of the invention are useful for treating any CNS condition involving inflammation, i.e., reduce the clinical symptoms of inflammation, including, but not limited to, ischemic stroke, multiple sclerosis, Alzheimer's Disease, ALS, Parkinson's Disease, hypoxic-ischemia, neonatal hypoxic ischemia, and traumatic brain or spinal cord injury.

In one particular embodiment, the methods and compositions of the invention are used to treat traumatic brain injury or hypoxic ischemia. For this treatment, one would administer the cells that modulate microglia activation. Such cells would have been assessed for the capacity to modulate microglia activation and selected for a desired degree of modulation.

It is understood, however, that for treatment of any of the above conditions, it may be expedient to use such cells; that is, one that has been assessed for modulating microglia activation and selected for a desired level of modulation prior to administration for treatment of the condition.

In a highly specific embodiment, the pathology is traumatic brain injury or spinal cord injury and the cells are non-embryonic stem, non-germ cells that have extended replicative capacity and pluripotentiality and, therefore, express certain markers, e.g., one or more of telomerase, oct4, rex-1, and rox-1, and have broad differentiation potential, e.g., at least two of ectodermal, endodermal, and mesodermal cell types.

### BRIEF DESCRIPTION OF THE FIGURES

FIGS. 1A-C—Microglial stimulation by LPS in the presence of MultiStem® co-culture prevents microglial upregulation of proinflammatory molecules. BV2 mouse microglial cells were plated at 1×10⁵ cells/ml, in the presence of human MultiStem® added at 2×10⁵ cells/ml via TransWells. Control wells received TransWells with no MultiStem® added. Co-cultures were maintained for 24 hours, followed by 24 hours stimulation with LPS (5 μg/ml). Media was collected from the co-cultures and analyzed by ELISAs specific for mouse TNF-α (FIG. 1A), IL-6 (FIG. 1B), and MMP-9 (FIG. 1C) (R&D Systems, Inc.).

FIGS. 2A-C—Pre-incubation of microglial cells with MultiStem® results in prolonged inhibition of microglial activation potential as measured by release of proinflammatory molecules after stimulation with LPS. BV2 mouse microglial cells were plated at 1×10⁵ cells/ml, in the presence of human MultiStem® added at 2×10⁵ cells/ml via TransWells. Control wells received TransWells with no MultiStem® added. Co-cultures were maintained for 24 hours, after which the TransWells (containing MultiStem®) were discarded. Microglia were washed 3× with PBS, and fed with fresh media. After 3 hours, microglia were stimulated with LPS (5 μg/ml). After 24 hours LPS stimulation, media was collected from the microglia and analyzed by ELISAs specific for mouse TNF-α (FIG. 2A), IL-6 (FIG. 2B), and MMP-9 (FIG. 2C).

FIGS. 3A-C—Treatment of microglial cells with conditioned media from MultiStem® inhibits microglial release of proinflammatory molecules in response to LPS stimulation. BV2 mouse microglial cells were plated at 1×10⁵ cells/ml. The media on the microglial cells was replaced with either MultiStem® basal media (controls), or conditioned media collected from nascent human MultiStem® cultures. After 3 hours, the microglia were stimulated with LPS (5 μg/ml), in the presence of the basal or conditioned medias. After 24 hours LPS stimulation, media was collected from the microglial cells and analyzed by ELISAs specific for mouse TNF-α (FIG. 3A), IL-6 (FIG. 3B), and MMP-9 (FIG. 3C).

FIG. 4—MultiStem® significantly inhibits ischemia-induced markers of immune response within infarct region. qPCR analysis confirms that MultiStem® administration prevents massive ischemia-induced upregulation of markers of immune response, such as CD8a, Gal-3, and MMP12 (expressed by T cells, and microglia) (left panel). qPCR analysis of different brain regions demonstrates that the changes in immune marker genes are specific to the infarct region (right panel).

### DETAILED DESCRIPTION OF THE INVENTION

It should be understood that this invention is not limited to the particular methodology, protocols, and reagents, etc., described herein and, as such, may vary. The terminology used herein is for the purpose of describing particular embodiments only, and is not intended to limit the scope of the disclosed invention, which is defined solely by the claims.

The section headings are used herein for organizational purposes only and are not to be construed as in any way limiting the subject matter described.

The methods and techniques of the present application are generally performed according to conventional methods well-known in the art and as described in various general and more specific references that are cited and discussed throughout the present specification unless otherwise indicated. See, e.g., Sambrook et al., Molecular Cloning: A Laboratory Manual, 3rd ed., Cold Spring Harbor Laboratory Press, Cold Spring Harbor, N.Y. (2001) and Ausubel et al., Current Protocols in Molecular Biology, Greene Publishing Associates (1992), and Harlow and Lane, Antibodies: A Laboratory Manual, Cold Spring Harbor Laboratory Press, Cold Spring Harbor, N.Y. (1990).

### Definitions

“A” or “an” means herein one or more than one; at least one. Where the plural form is used herein, it generally includes the singular.

A “cell bank” is industry nomenclature for cells that have been grown and stored for future use. Cells may be stored in aliquots. They can be used directly out of storage or may be expanded after storage. This is a convenience so that there are “off the shelf” cells available for administration. The cells may already be stored in a pharmaceutically-acceptable excipient so they may be directly administered or they may be mixed with an appropriate excipient when they are released from storage. Cells may be frozen or otherwise stored in a form to preserve viability. In one embodiment of the invention, cell banks are created in which the cells have been selected for enhanced modulation of activation of microglia. Following release from storage, and prior to administration to the subject, it may be preferable to again assay the cells for potency, i.e., level of modulation of activation of microglia. This can be done using any of the assays, direct or indirect, described in this application or otherwise known in the art. Then cells having the desired potency can then be administered to the subject for treatment. Banks can be made using cells derived from the individual to be treated (from their pre-natal tissues such as placenta, umbilical cord blood, or umbilical cord matrix or expanded from the individual at any time after birth) (autologous). Or banks can contain cells for allogeneic uses.

“Co-administer” means to administer in conjunction with one another, together, coordinately, including simultaneous or sequential administration of two or more agents.

“Comprising” means, without other limitation, including the referent, necessarily, without any qualification or exclusion on what else may be included. For example, “a composition comprising x and y” encompasses any composition that contains x and y, no matter what other components may be present in the composition. Likewise, “a method comprising the step of x” encompasses any method in which x is carried out, whether x is the only step in the method or it is only one of the steps, no matter how many other steps there may be and no matter how simple or complex x is in comparison to them. “Comprised of and similar phrases using words of the root “comprise” are used herein as synonyms of “comprising” and have the same meaning.

“Comprised of” is a synonym of “comprising” (see above).

“Conditioned cell culture medium” is a term well-known in the art and refers to medium in which cells have been grown. Herein this means that the cells are grown for a sufficient time to secrete the factors that are effective to achieve any of the results described in this application, including modulating microglia activation, etc.

Conditioned cell culture medium refers to medium in which cells have been cultured so as to secrete factors into the medium. For the purposes of the present invention, cells can be grown through a sufficient number of cell divisions so as to produce effective amounts of such factors so that the medium has the effects, including modulating microglia activation, etc. Cells are removed from the medium by any of the known methods in the art, including, but not limited to, centrifugation, filtration, immunodepletion (e.g., via tagged antibodies and magnetic columns), and FACS sorting.

“EC cells” were discovered from analysis of a type of cancer called a teratocarcinoma. In 1964, researchers noted that a single cell in teratocarcinomas could be isolated and remain undifferentiated in culture. This type of stem cell became known as an embryonic carcinoma cell (EC cell).

“Effective amount” generally means an amount which provides the desired local or systemic effect, e.g., effective to ameliorate undesirable effects of inflammation, including modulating microglia activation, etc. For example, an effective amount is an amount sufficient to effectuate a beneficial or desired clinical result. The effective amounts can be provided all at once in a single administration or in fractional amounts that provide the effective amount in several administrations. The precise determination of what would be considered an effective amount may be based on factors individual to each subject, including their size, age, injury, and/or disease or injury being treated, and amount of time since the injury occurred or the disease began. One skilled in the art will be able to determine the effective amount for a given subject based on these considerations which are routine in the art. As used herein, “effective dose” means the same as “effective amount.”

“Effective route” generally means a route which provides for delivery of an agent to a desired compartment, system, or location. For example, an effective route is one through which an agent can be administered to provide at the desired site of action an amount of the agent sufficient to effectuate a beneficial or desired clinical result.

“Embryonic Stem Cells (ESC)” are well known in the art and have been prepared from many different mammalian species. Embryonic stem cells are stem cells derived from the inner cell mass of an early stage embryo known as a blastocyst. They are able to differentiate into all derivatives of the three primary germ layers: ectoderm, endoderm, and mesoderm. These include each of the more than 220 cell types in the adult body. The ES cells can become any tissue in the body, excluding placenta. Only the morula's cells are totipotent, able to become all tissues and a placenta. Some cells similar to ESCs may be produced by nuclear transfer of a somatic cell nucleus into an enucleated fertilized egg.

Use of the term “includes” is not intended to be limiting.

“Increase” or “increasing” means to induce a biological event entirely or to increase the degree of the event. With respect to increasing the neuroprotective activation state, it means to induce it entirely where there was no pre-existing activation or to increase the degree of neuroprotective activation.

“Induced pluripotent stem cells (IPSC or IPS cells)” are somatic cells that have been reprogrammed. For example, by introducing exogenous genes that confer on the somatic cell a less differentiated phenotype. These cells can then be induced to differentiate into less differentiated progeny. IPS cells have been derived using modifications of an approach originally discovered in 2006 (Yamanaka, S. et al., Cell Stem Cell, 1:39-49 (2007)). For example, in one instance, to create IPS cells, scientists started with skin cells that were then modified by a standard laboratory technique using retroviruses to insert genes into the cellular DNA. In one instance, the inserted genes were Oct4, Sox2, Lif4, and c-myc, known to act together as natural regulators to keep cells in an embryonic stem cell-like state. These cells have been described in the literature. See, for example, Wernig et al., PNAS, 105:5856-5861 (2008); Jaenisch et al., Cell, 132:567-582 (2008); Hanna et al., Cell, 133:250-264 (2008); and Brambrink et al., Cell Stem Cell, 2:151-159 (2008). These references are incorporated by reference for teaching IPSCs and methods for producing them. It is also possible that such cells can be created by specific culture conditions (exposure to specific agents).

The term “isolated” refers to a cell or cells which are not associated with one or more cells or one or more cellular components that are associated with the cell or cells in vivo. An “enriched population” means a relative increase in numbers of a desired cell relative to one or more other cell types in vivo or in primary culture.

However, as used herein, the term “isolated” does not indicate the presence of only the cells of the invention. Rather, the term “isolated” indicates that the cells of the invention are removed from their natural tissue environment and are present at a higher concentration as compared to the normal tissue environment. Accordingly, an “isolated” cell population may further include cell types in addition to the cells of the invention and may include additional tissue components. This also can be expressed in terms of cell doublings, for example. A cell may have undergone 10, 20, 30, 40 or more doublings in vitro or ex vivo so that it is enriched compared to its original numbers in vivo or in its original tissue environment (e.g., bone marrow, peripheral blood, adipose tissue, etc.).

“MAPC” is an acronym for “multipotent adult progenitor cell.” It refers to a cell that is not an embryonic stem cell or germ cell but has some characteristics of these. MAPC can be characterized in a number of alternative descriptions, each of which conferred novelty to the cells when they were discovered. They can, therefore, be characterized by one or more of those descriptions. First, they have extended replicative capacity in culture without being transformed (tumorigenic) and with a normal karyotype. Second, they may give rise to cell progeny of more than one germ layer, such as two or all three germ layers (i.e., endoderm, mesoderm and ectoderm) upon differentiation. Third, although they are not embryonic stem cells or germ cells, they may express markers of these primitive cell types so that MAPCs may express one or more of Oct 3/4 (i.e., Oct 3A), rex-1, and rox-1. They may also express one or more of sox-2 and SSEA-4. Fourth, like a stem cell, they may self-renew, that is, have an extended replication capacity without being transformed. This means that these cells express telomerase (i.e., have telomerase activity). Accordingly, the cell type that was designated “MAPC” may be characterized by alternative basic characteristics that describe the cell via some of its novel properties.

The term “adult” in MAPC is non-restrictive. It refers to a non-embryonic somatic cell. MAPCs are karyotypically normal and do not form teratomas in vivo. This acronym was first used in U.S. Pat. No. 7,015,037 to describe a pluripotent cell isolated from bone marrow. However, cells with pluripotential markers and/or differentiation potential have been discovered subsequently and, for purposes of this invention, may be equivalent to those cells first designated “MAPC.” Essential descriptions of the MAPC type of cell are provided in the Summary of the Invention above.

MAPC represents a more primitive progenitor cell population than MSC (Verfaillie, C. M., Trends Cell Biol 12:502-8 (2002), Jahagirdar, B. N., et al., Exp Hematol, 29:543-56 (2001); Reyes, M. and C. M. Verfaillie, Ann N Y Acad Sci, 938:231-233 (2001); Jiang, Y. et al., Exp Hematol, 30896-904 (2002); and (Jiang, Y. et al., Nature, 418:41-9. (2002)).

The term “MultiStem®” is the trade name for a cell preparation based on the MAPCs of U.S. Pat. No. 7,015,037, i.e., a non-embryonic stem, non-germ cell as described above. MultiStem® is prepared according to cell culture methods disclosed in this patent application, particularly, lower oxygen and higher serum.

“Pharmaceutically-acceptable carrier” is any pharmaceutically-acceptable medium for the cells used in the present invention. Such a medium may retain isotonicity, cell metabolism, pH, and the like. It is compatible with administration to a subject in vivo, and can be used, therefore, for cell delivery and treatment.

The term “potency” refers to the ability of the cells (or conditioned medium from the cells) to achieve the various effects described in this application. Accordingly, potency refers to the effect at various levels, including, but not limited to, reducing symptoms of inflammation, modulating microglia activation, etc.

“Primordial embryonic germ cells” (PG or EG cells) can be cultured and stimulated to produce many less differentiated cell types.

“Progenitor cells” are cells produced during differentiation of a stem cell that have some, but not all, of the characteristics of their terminally-differentiated progeny. Defined progenitor cells, such as “cardiac progenitor cells,” are committed to a lineage, but not to a specific or terminally differentiated cell type. The term “progenitor” as used in the acronym “MAPC” does not limit these cells to a particular lineage. A progenitor cell can form a progeny cell that is more highly differentiated than the progenitor cell.

The term “reduce” as used herein means to prevent as well as decrease. In the context of treatment, to “reduce” is to either prevent or ameliorate one or more clinical symptoms. A clinical symptom is one (or more) that has or will have, if left untreated, a negative impact on the quality of life (health) of the subject. This also applies to the underlying biological effects such as reducing pro-inflammatory molecules, activation of microglia, etc., the end result of which would be to ameliorate the deleterious effects of inflammation.

“Selecting” a cell with a desired level of potency (e.g., for modulating microglia activation) can mean identifying (as by assay), isolating, and expanding a cell. This could create a population that has a higher potency than the parent cell population from which the cell was isolated. The “parent” cell population refers to the parent cells from which the selected cells are divided. “Parent” refers to an actual P1→F1 relationship (i.e., a cell progeny). So if cell X is isolated from a mixed population of cells X and Y, in which X is an expressor and Y is not, one would not classify a mere isolate of X as having enhanced expression. But, if a progeny cell of X is a higher expressor, one would classify the progeny cell as having enhanced expression.

To select a cell that modulates microglia activation would include both an assay to determine if there is modulation and would also include obtaining that cell. The cell may naturally modulate microglia activation in that the cell was not incubated with or exposed to an agent that induces modulation of activation. The cell may not be known to be a modulator of microglia activation prior to conducting the assay. As modulation could depend on gene expression and/or secretion, one could also select on the basis of one or more of the genes that cause modulation.

Selection could be from cells in a tissue. For example, in this case, cells would be isolated from a desired tissue, expanded in culture, selected for modulation of microglia activation, and the selected cells further expanded.

Selection could also be from cells ex vivo, such as cells in culture. In this case, one or more of the cells in culture would be assayed for modulation of activation and the cells obtained that modulate the activation could be further expanded.

Cells could also be selected for enhanced modulation of activation. In this case, the cell population from which the enhanced cell is obtained already modulates the activation. Enhanced modulation means a higher average amount per cell than in the parent population.

The parent population from which the enhanced cell is selected may be substantially homogeneous (the same cell type). One way to obtain such an enhanced cell from this population is to create single cells or cell pools and assay those cells or cell pools for modulation of activation to obtain clones that naturally modulate the activation (as opposed to treating the cells with a modulator or inducer of activation) and then expanding those cells that are naturally enhanced.

However, cells may be treated with one or more agents that will enhance modulation of activation of microglia via the endogenous cellular pathways. Thus, substantially homogeneous populations may be treated to enhance modulation.

If the population is not substantially homogeneous, then, it is preferable that the parental cell population to be treated contains at least 100 of the modulator cell type in which enhanced modulation is sought, more preferably at least 1,000 of the cells, and still more preferably, at least 10,000 of the cells. Following treatment, this sub-population can be recovered from the heterogeneous population by known cell selection techniques and further expanded if desired.

Thus, desired levels of modulation of activation may be those that are higher than the levels in a given preceding population. For example, cells that are put into primary culture from a tissue and expanded and isolated by culture conditions that are not specifically designed to promote modulation of activation, may provide a parent population. Such a parent population can be treated to enhance the average modulation per cell or screened for a cell or cells within the population that express higher modulation without deliberate treatment. Such cells can be expanded then to provide a population with a higher (desired) expression.

“Self-renewal” refers to the ability to produce replicate daughter stem cells having differentiation potential that is identical to those from which they arose. A similar term used in this context is “proliferation.”

“Stem cell” means a cell that can undergo self-renewal (i.e., progeny with the same differentiation potential) and also produce progeny cells that are more restricted in differentiation potential. Within the context of the invention, a stem cell would also encompass a more differentiated cell that has de-differentiated, for example, by nuclear transfer, by fusion with a more primitive stem cell, by introduction of specific transcription factors, or by culture under specific conditions. See, for example, Wilmut et al., Nature, 385:810-813 (1997); Ying et al., Nature, 416:545-548 (2002); Guan et al., Nature, 440:1199-1203 (2006); Takahashi et al., Cell, 126:663-676 (2006); Okita et al., Nature, 448:313-317 (2007); and Takahashi et al., Cell, 131:861-872 (2007).

Dedifferentiation may also be caused by the administration of certain compounds or exposure to a physical environment in vitro or in vivo that would cause the dedifferentiation. Stem cells also may be derived from abnormal tissue, such as a teratocarcinoma and some other sources such as embryoid bodies (although these can be considered embryonic stem cells in that they are derived from embryonic tissue, although not directly from the inner cell mass). Stem cells may also be produced by introducing genes associated with stem cell function into a non-stem cell, such as an induced pluripotent stem cell.

“Subject” means a vertebrate, such as a mammal, such as a human. Mammals include, but are not limited to, humans, dogs, cats, horses, cows, and pigs.

The term “therapeutically effective amount” refers to the amount of an agent determined to produce any therapeutic response in a mammal. For example, effective anti-inflammatory therapeutic agents may prolong the survivability of the patient, and/or inhibit overt clinical symptoms. Treatments that are therapeutically effective within the meaning of the term as used herein, include treatments that improve a subject's quality of life even if they do not improve the disease outcome per se. Such therapeutically effective amounts are readily ascertained by one of ordinary skill in the art. Thus, to “treat” means to deliver such an amount. Thus, treating can prevent or ameliorate any pathological symptoms of inflammation.

“Treat,” “treating,” or “treatment” are used broadly in relation to the invention and each such term encompasses, among others, preventing, ameliorating, inhibiting, or curing a deficiency, dysfunction, disease, or other deleterious process, including those that interfere with and/or result from a therapy.

“Validate” means to confirm. In the context of the invention, one confirms that a cell is an expressor with a desired potency. This is so that one can then use that cell (in treatment, banking, drug screening, etc.) with a reasonable expectation of efficacy. Accordingly, to validate means to confirm that the cells, having been originally found to have/established as having the ability to modulate microglia activation, in fact, retain that ability. Thus, validation is a verification event in a two-event process involving the original determination and the follow-up determination. The second event is referred to herein as “validation.”

### Stem Cells

The present invention can be practiced, preferably, using stem cells of vertebrate species, such as humans, non-human primates, domestic animals, livestock, and other non-human mammals. These include, but are not limited to, those cells described below.

Embryonic Stem Cells

The most well studied stem cell is the embryonic stem cell (ESC) as it has unlimited self-renewal and multipotent differentiation potential. These cells are derived from the inner cell mass of the blastocyst or can be derived from the primordial germ cells of a post-implantation embryo (embryonal germ cells or EG cells). ES and EG cells have been derived, first from mouse, and later, from many different animals, and more recently, also from non-human primates and humans. When introduced into mouse blastocysts or blastocysts of other animals, ESCs can contribute to all tissues of the animal. ES and EG cells can be identified by positive staining with antibodies against SSEA1 (mouse) and SSEA4 (human). See, for example, U.S. Pat. Nos. 5,453,357; 5,656,479; 5,670,372; 5,843,780; 5,874,301; 5,914,268; 6,110,739 6,190,910; 6,200,806; 6,432,711; 6,436,701, 6,500,668; 6,703,279; 6,875,607; 7,029,913; 7,112,437; 7,145,057; 7,153,684; and 7,294,508, each of which is incorporated by reference for teaching embryonic stem cells and methods of making and expanding them. Accordingly, ESCs and methods for isolating and expanding them are well-known in the art.

A number of transcription factors and exogenous cytokines have been identified that influence the potency status of embryonic stem cells in vivo. The first transcription factor to be described that is involved in stem cell pluripotency is Oct4. Oct4 belongs to the POU (Pit-Oct-Unc) family of transcription factors and is a DNA binding protein that is able to activate the transcription of genes, containing an octameric sequence called “the octamer motif” within the promoter or enhancer region. Oct4 is expressed at the moment of the cleavage stage of the fertilized zygote until the egg cylinder is formed. The function of Oct3/4 is to repress differentiation inducing genes (i.e., FoxaD3, hCG) and to activate genes promoting pluripotency (FGF4, Utf1, Rex1). Sox2, a member of the high mobility group (HMG) box transcription factors, cooperates with Oct4 to activate transcription of genes expressed in the inner cell mass. It is essential that Oct3/4 expression in embryonic stem cells is maintained between certain levels. Overexpression or downregulation of >50% of Oct4 expression level will alter embryonic stem cell fate, with the formation of primitive endoderm/mesoderm or trophectoderm, respectively. In vivo, Oct4 deficient embryos develop to the blastocyst stage, but the inner cell mass cells are not pluripotent. Instead they differentiate along the extraembryonic trophoblast lineage. Sall4, a mammalian Spalt transcription factor, is an upstream regulator of Oct4, and is therefore important to maintain appropriate levels of Oct4 during early phases of embryology. When Sall4 levels fall below a certain threshold, trophectodermal cells will expand ectopically into the inner cell mass. Another transcription factor required for pluripotency is Nanog, named after a celtic tribe “Tir Nan Og”: the land of the ever young. In vivo, Nanog is expressed from the stage of the compacted morula, is subsequently defined to the inner cell mass and is downregulated by the implantation stage. Downregulation of Nanog may be important to avoid an uncontrolled expansion of pluripotent cells and to allow multilineage differentiation during gastrulation. Nanog null embryos, isolated at day 5.5, consist of a disorganized blastocyst, mainly containing extraembryonic endoderm and no discernable epiblast.

Non-Embryonic Stem Cells

Stem cells have been identified in most tissues. Perhaps the best characterized is the hematopoietic stem cell (HSC). HSCs are mesoderm-derived cells that can be purified using cell surface markers and functional characteristics. They have been isolated from bone marrow, peripheral blood, cord blood, fetal liver, and yolk sac. They initiate hematopoiesis and generate multiple hematopoietic lineages. When transplanted into lethally-irradiated animals, they can repopulate the erythroid neutrophil-macrophage, megakaryocyte, and lymphoid hematopoietic cell pool. They can also be induced to undergo some self-renewal cell division. See, for example, U.S. Pat. Nos. 5,635,387; 5,460,964; 5,677,136; 5,750,397; 5,681,599; and 5,716,827. U.S. Pat. No. 5,192,553 reports methods for isolating human neonatal or fetal hematopoietic stem or progenitor cells. U.S. Pat. No. 5,716,827 reports human hematopoietic cells that are Thy-1⁺ progenitors, and appropriate growth media to regenerate them in vitro. U.S. Pat. No. 5,635,387 reports a method and device for culturing human hematopoietic cells and their precursors. U.S. Pat. No. 6,015,554 describes a method of reconstituting human lymphoid and dendritic cells. Accordingly, HSCs and methods for isolating and expanding them are well-known in the art.

Another stem cell that is well-known in the art is the neural stem cell (NSC). These cells can proliferate in vivo and continuously regenerate at least some neuronal cells. When cultured ex vivo, neural stem cells can be induced to proliferate as well as differentiate into different types of neurons and glial cells. When transplanted into the brain, neural stem cells can engraft and generate neural and glial cells. See, for example, Gage F. H., Science, 287:1433-1438 (2000), Svendsen S. N. et al, Brain Pathology, 9:499-513 (1999), and Okabe S. et al., Mech Development, 59:89-102 (1996). U.S. Pat. No. 5,851,832 reports multipotent neural stem cells obtained from brain tissue. U.S. Pat. No. 5,766,948 reports producing neuroblasts from newborn cerebral hemispheres. U.S. Pat. Nos. 5,564,183 and 5,849,553 report the use of mammalian neural crest stem cells. U.S. Pat. No. 6,040,180 reports in vitro generation of differentiated neurons from cultures of mammalian multipotential CNS stem cells. WO 98/50526 and WO 99/01159 report generation and isolation of neuroepithelial stem cells, oligodendrocyte-astrocyte precursors, and lineage-restricted neuronal precursors. U.S. Pat. No. 5,968,829 reports neural stem cells obtained from embryonic forebrain. Accordingly, neural stem cells and methods for making and expanding them are well-known in the art.

Another stem cell that has been studied extensively in the art is the mesenchymal stem cell (MSC). MSCs are derived from the embryonal mesoderm and can be isolated from many sources, including adult bone marrow, peripheral blood, fat, placenta, and umbilical blood, among others. MSCs can differentiate into many mesodermal tissues, including muscle, bone, cartilage, fat, and tendon. There is considerable literature on these cells. See, for example, U.S. Pat. Nos. 5,486,389; 5,827,735; 5,811,094; 5,736,396; 5,837,539; 5,837,670; and 5,827,740. See also Pittenger, M. et al, Science, 284:143-147 (1999).

Another example of an adult stem cell is adipose-derived adult stem cells (ADSCs) which have been isolated from fat, typically by liposuction followed by release of the ADSCs using collagenase. ADSCs are similar in many ways to MSCs derived from bone marrow, except that it is possible to isolate many more cells from fat. These cells have been reported to differentiate into bone, fat, muscle, cartilage, and neurons. A method of isolation has been described in U.S. 2005/0153442.

Other stem cells that are known in the art include gastrointestinal stem cells, epidermal stem cells, and hepatic stem cells, which have also been termed “oval cells” (Potten, C., et al., Trans R Soc Lond B Biol Sci, 353:821-830 (1998), Watt, F., Trans R Soc Lond B Biol Sci, 353:831 (1997); Alison et al., Hepatology, 29:678-683 (1998).

Other non-embryonic cells reported to be capable of differentiating into cell types of more than one embryonic germ layer include, but are not limited to, cells from umbilical cord blood (see U.S. Publication No. 2002/0164794), placenta (see U.S. Publication No. 2003/0181269, umbilical cord matrix (Mitchell, K. E. et al., Stem Cells, 21:50-60 (2003)), small embryonic-like stem cells (Kucia, M. et al., J Physiol Pharmacol, 57 Suppl 5:5-18 (2006)), amniotic fluid stem cells (Atala, A., J Tissue Regen Med, 1:83-96 (2007)), skin-derived precursors (Toma et al., Nat Cell Biol, 3:778-784 (2001)), and bone marrow (see U.S. Publication Nos. 2003/0059414 and 2006/0147246), each of which is incorporated by reference for teaching these cells.

Strategies of Reprogramming Somatic Cells

Several different strategies such as nuclear transplantation, cellular fusion, and culture induced reprogramming have been employed to induce the conversion of differentiated cells into an embryonic state. Nuclear transfer involves the injection of a somatic nucleus into an enucleated oocyte, which, upon transfer into a surrogate mother, can give rise to a clone (“reproductive cloning”), or, upon explantation in culture, can give rise to genetically matched embryonic stem (ES) cells (“somatic cell nuclear transfer,” SCNT). Cell fusion of somatic cells with ES cells results in the generation of hybrids that show all features of pluripotent ES cells. Explantation of somatic cells in culture selects for immortal cell lines that may be pluripotent or multipotent. At present, spermatogonial stem cells are the only source of pluripotent cells that can be derived from postnatal animals. Transduction of somatic cells with defined factors can initiate reprogramming to a pluripotent state. These experimental approaches have been extensively reviewed (Hochedlinger and Jaenisch, Nature, 441:1061-1067 (2006) and Yamanaka, S., Cell Stem Cell, 1:39-49 (2007)).

Nuclear Transfer

Nuclear transplantation (NT), also referred to as somatic cell nuclear transfer (SCNT), denotes the introduction of a nucleus from a donor somatic cell into an enucleated ogocyte to generate a cloned animal such as Dolly the sheep (Wilmut et al., Nature, 385:810-813 (1997). The generation of live animals by NT demonstrated that the epigenetic state of somatic cells, including that of terminally differentiated cells, while stable, is not irreversible fixed but can be reprogrammed to an embryonic state that is capable of directing development of a new organism. In addition to providing an exciting experimental approach for elucidating the basic epigenetic mechanisms involved in embryonic development and disease, nuclear cloning technology is of potential interest for patient-specific transplantation medicine.

Fusion of Somatic Cells and Embryonic Stem Cells

Epigenetic reprogramming of somatic nuclei to an undifferentiated state has been demonstrated in murine hybrids produced by fusion of embryonic cells with somatic cells. Hybrids between various somatic cells and embryonic carcinoma cells (Solter, D., Nat Rev Genet, 7:319-327 (2006), embryonic germ (EG), or ES cells (Zwaka and Thomson, Development, 132:227-233 (2005)) share many features with the parental embryonic cells, indicating that the pluripotent phenotype is dominant in such fusion products. As with mouse (Tada et al., Curr Biol, 11:1553-1558 (2001)), human ES cells have the potential to reprogram somatic nuclei after fusion (Cowan et al., Science, 309:1369-1373(2005)); Yu et al., Science, 318:1917-1920 (2006)). Activation of silent pluripotency markers such as Oct4 or reactivation of the inactive somatic X chromosome provided molecular evidence for reprogramming of the somatic genome in the hybrid cells. It has been suggested that DNA replication is essential for the activation of pluripotency markers, which is first observed 2 days after fusion (Do and Scholer, Stem Cells, 22:941-949 (2004)), and that forced overexpression of Nanog in ES cells promotes pluripotency when fused with neural stem cells (Silva et al., Nature, 441:997-1001 (2006)).

Culture-Induced Reprogramming

Pluripotent cells have been derived from embryonic sources such as blastomeres and the inner cell mass (ICM) of the blastocyst (ES cells), the epiblast (EpiSC cells), primordial germ cells (EG cells), and postnatal spermatogonial stem cells (“maGSCsm” “ES-like” cells). The following pluripotent cells, along with their donor cell/tissue is as follows: parthogenetic ES cells are derived from murine oocytes (Narasimha et al., Curr Biol, 7:881-884 (1997)); embryonic stem cells have been derived from blastomeres (Wakayama et al., Stem Cells, 25:986-993 (2007)); inner cell mass cells (source not applicable) (Eggan et al., Nature, 428:44-49 (2004)); embryonic germ and embryonal carcinoma cells have been derived from primordial germ cells (Matsui et al., Cell, 70:841-847 (1992)); GMCS, maSSC, and MASC have been derived from spermatogonial stem cells (Guan et al., Nature, 440:1199-1203 (2006); Kanatsu-Shinohara et al., Cell, 119:1001-1012 (2004); and Seandel et al., Nature, 449:346-350 (2007)); EpiSC cells are derived from epiblasts (Brons et al., Nature, 448:191-195 (2007); Tesar et al., Nature, 448:196-199(2007)); parthogenetic ES cells have been derived from human oocytes (Cibelli et al., Science, 295L819 (2002); Revazova et al., Cloning Stem Cells, 9:432-449 (2007)); human ES cells have been derived from human blastocysts (Thomson et al., Science, 282:1145-1147 (1998)); MAPC have been derived from bone marrow (Jiang et al., Nature, 418:41-49 (2002); Phinney and Prockop, Stem Cells, 25:2896-2902 (2007)); cord blood cells (derived from cord blood) (van de Ven et al., Exp Hematol, 35:1753-1765 (2007)); neurosphere derived cells derived from neural cell (Clarke et al., Science, 288:1660-1663 (2000)). Donor cells from the germ cell lineage such as PGCs or spermatogonial stem cells are known to be unipotent in vivo, but it has been shown that pluripotent ES-like cells (Kanatsu-Shinohara et al., Cell, 119:1001-1012 (2004) or maGSCs (Guan et al., Nature, 440:1199-1203 (2006), can be isolated after prolonged in vitro culture. While most of these pluripotent cell types were capable of in vitro differentiation and teratoma formation, only ES, EG, EC, and the spermatogonial stem cell-derived maGCSs or ES-like cells were pluripotent by more stringent criteria, as they were able to form postnatal chimeras and contribute to the germline Recently, multipotent adult spermatogonial stem cells (MASCs) were derived from testicular spermatogonial stem cells of adult mice, and these cells had an expression profile different from that of ES cells (Seandel et al., Nature, 449:346-350 (2007)) but similar to EpiSC cells, which were derived from the epiblast of postimplantation mouse embryos (Brons et al., Nature, 448:191-195 (2007); Tesar et al., Nature, 448:196-199 (2007)).

Reprogramming by Defined Transcription Factors

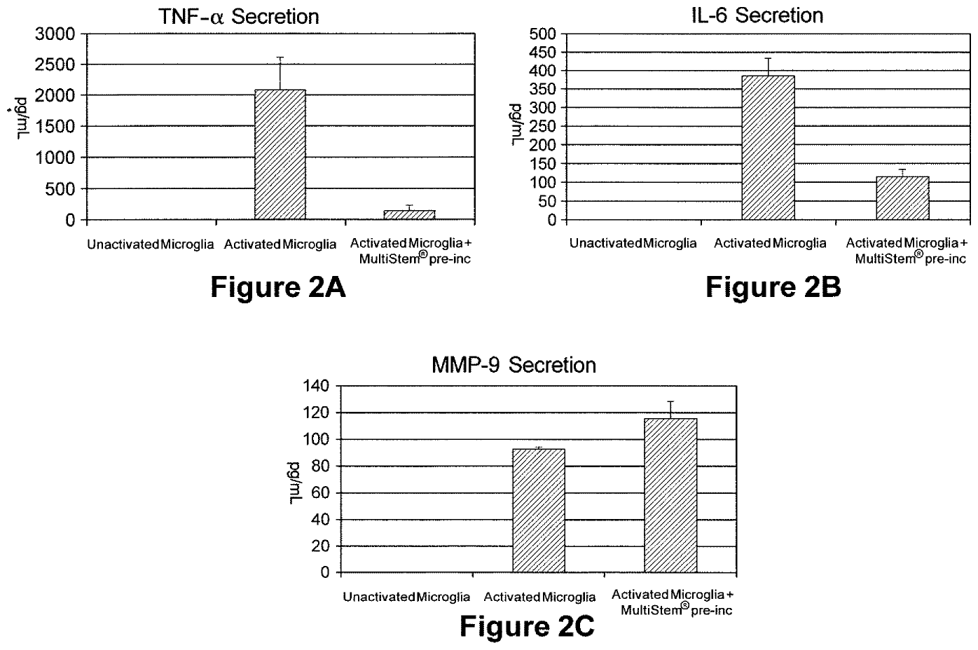

Takahashi and Yamanaka have reported reprogramming somatic cells back to an ES-like state (Takahashi and Yamanaka, Cell, 126:663-676 (2006)). They successfully reprogrammed mouse embryonic fibroblasts (MEFs) and adult fibroblasts to pluripotent ES-like cells after viral-mediated transduction of the four transcription factors Oct4, Sox2, c-myc, and Klf4 followed by selection for activation of the Oct4 target gene Fbx15 (FIG. 2A). Cells that had activated Fbx15 were coined iPS (induced pluripotent stem) cells and were shown to be pluripotent by their ability to form teratomas, although the were unable to generate live chimeras. This pluripotent state was dependent on the continuous viral expression of the transduced Oct4 and Sox2 genes, whereas the endogenous Oct4 and Nanog genes were either not expressed or were expressed at a lower level than in ES cells, and their respective promoters were found to be largely methylated. This is consistent with the conclusion that the Fbx15-iPS cells did not correspond to ES cells but may have represented an incomplete state of reprogramming. While genetic experiments had established that Oct4 and Sox2 are essential for pluripotency (Chambers and Smith, Oncogene, 23:7150-7160 (2004); Ivanona et al., Nature, 442:5330538 (2006); Masui et al., Nat Cell Biol, 9:625-635 (2007)), the role of the two oncogenes c-myc and Klf4 in reprogramming is less clear. Some of these oncogenes may, in fact, be dispensable for reprogramming, as both mouse and human iPS cells have been obtained in the absence of c-myc transduction, although with low efficiency (Nakagawa et al., Nat Biotechnol, 26:191-106 (2008); Werning et al., Nature, 448:318-324 (2008); Yu et al., Science, 318: 1917-1920 (2007)).

### MAPC

Human MAPCs are described in U.S. Pat. No. 7,015,037. MAPCs have been identified in other mammals. Murine MAPCs, for example, are also described in U.S. Pat. No. 7,015,037. Rat MAPCs are also described in U.S. Pat. No. 7,838,289.

These references are incorporated by reference for describing MAPCs first isolated by Catherine Verfaillie.

### Isolation and Growth of MAPCs

Methods of MAPC isolation are known in the art. See, for example, U.S. Pat. No. 7,015,037, and these methods, along with the characterization (phenotype) of MAPCs, are incorporated herein by reference. MAPCs can be isolated from multiple sources, including, but not limited to, bone marrow, placenta, umbilical cord and cord blood, muscle, brain, liver, spinal cord, blood or skin. It is, therefore, possible to obtain bone marrow aspirates, brain or liver biopsies, and other organs, and isolate the cells using positive or negative selection techniques available to those of skill in the art, relying upon the genes that are expressed (or not expressed) in these cells (e.g., by functional or morphological assays such as those disclosed in the above-referenced applications, which have been incorporated herein by reference).

MAPCs have also been obtained my modified methods described in Breyer et al., Experimental Hematology, 34:1596-1601 (2006) and Subramanian et al., Cellular Programming and Reprogramming Methods and Protocols; S. Ding (ed.), Methods in Molecular Biology, 636:55-78 (2010), incorporated by reference for these methods.

MAPCs from Human Bone Marrow as Described in U.S. Pat. No. 7,015,037

MAPCs do not express the common leukocyte antigen CD45 or erythroblast specific glycophorin-A (Gly-A). The mixed population of cells was subjected to a Ficoll Hypaque separation. The cells were then subjected to negative selection using anti-CD45 and anti-Gly-A antibodies, depleting the population of CD45⁺ and Gly-A⁺ cells, and the remaining approximately 0.1% of marrow mononuclear cells were then recovered. Cells could also be plated in fibronectin-coated wells and cultured as described below for 2-4 weeks to deplete the cells of CD45⁺ and Gly-A⁺ cells. In cultures of adherent bone marrow cells, many adherent stromal cells undergo replicative senescence around cell doubling 30 and a more homogenous population of cells continues to expand and maintains long telomeres.

Alternatively, positive selection could be used to isolate cells via a combination of cell-specific markers. Both positive and negative selection techniques are available to those of skill in the art, and numerous monoclonal and polyclonal antibodies suitable for negative selection purposes are also available in the art (see, for example, Leukocyte Typing V, Schlossman, et al., Eds. (1995) Oxford University Press) and are commercially available from a number of sources.

Techniques for mammalian cell separation from a mixture of cell populations have also been described by Schwartz, et al., in U.S. Pat. No. 5,759,793 (magnetic separation), Basch et al., 1983 (immunoaffinity chromatography), and Wysocki and Sato, 1978 (fluorescence-activated cell sorting).

Cells may be cultured in low-serum or serum-free culture medium. Serum-free medium used to culture MAPCs is described in U.S. Pat. No. 7,015,037. Commonly-used growth factors include but are not limited to platelet-derived growth factor and epidermal growth factor. See, for example, U.S. Pat. Nos. 7,169,610; 7,109,032; 7,037,721; 6,617,161; 6,617,159; 6,372,210; 6,224,860; 6,037,174; 5,908,782; 5,766,951; 5,397,706; and 4,657,866; all incorporated by reference for teaching growing cells in serum-free medium.

### Additional Culture Methods

In additional experiments the density at which MAPCs are cultured can vary from about 100 cells/cm² or about 150 cells/cm² to about 10,000 cells/cm², including about 200 cells/cm² to about 1500 cells/cm² to about 2000 cells/cm². The density can vary between species. Additionally, optimal density can vary depending on culture conditions and source of cells. It is within the skill of the ordinary artisan to determine the optimal density for a given set of culture conditions and cells.

Also, effective atmospheric oxygen concentrations of less than about 10%, including about 1-5% and, especially, 3-5%, can be used at any time during the isolation, growth and differentiation of MAPCs in culture.

Cells may be cultured under various serum concentrations, e.g., about 2-20%. Fetal bovine serum may be used. Higher serum may be used in combination with lower oxygen tensions, for example, about 15-20%. Cells need not be selected prior to adherence to culture dishes. For example, after a Ficoll gradient, cells can be directly plated, e.g., 250,000-500,000/cm². Adherent colonies can be picked, possibly pooled, and expanded.

In one embodiment, used in the experimental procedures in the Examples, high serum (around 15-20%) and low oxygen (around 3-5%) conditions were used for the cell culture. Specifically, adherent cells from colonies were plated and passaged at densities of about 1700-2300 cells/cm² in 18% serum and 3% oxygen (with PDGF and EGF).

In an embodiment specific for MAPCs, supplements are cellular factors or components that allow MAPCs to retain the ability to differentiate into cell types of more than one embryonic lineage, such as all three lineages. This may be indicated by the expression of specific markers of the undifferentiated state, such as Oct 3/4 (Oct 3A) and/or markers of high expansion capacity, such as telomerase.

### Cell Culture

For all the components listed below, see U.S. Pat. No. 7,015,037, which is incorporated by reference for teaching these components.

In general, cells useful for the invention can be maintained and expanded in culture medium that is available and well-known in the art. Also contemplated is supplementation of cell culture medium with mammalian sera. Additional supplements can also be used advantageously to supply the cells with the necessary trace elements for optimal growth and expansion. Hormones can also be advantageously used in cell culture. Lipids and lipid carriers can also be used to supplement cell culture media, depending on the type of cell and the fate of the differentiated cell. Also contemplated is the use of feeder cell layers.

Cells in culture can be maintained either in suspension or attached to a solid support, such as extracellular matrix components. Stem cells often require additional factors that encourage their attachment to a solid support, such as type I and type II collagen, chondroitin sulfate, fibronectin, “superfibronectin” and fibronectin-like polymers, gelatin, poly-D and poly-L-lysine, thrombospondin and vitronectin. One embodiment of the present invention utilizes fibronectin. See, for example, Ohashi et al., Nature Medicine, 13:880-885 (2007); Matsumoto et al., J Bioscience and Bioengineering, 105:350-354 (2008); Kirouac et al., Cell Stem Cell, 3:369-381 (2008); Chua et al., Biomaterials, 26:2537-2547 (2005); Drobinskaya et al., Stem Cells, 26:2245-2256 (2008); Dvir-Ginzberg et al., FASEB J, 22:1440-1449 (2008); Turner et al., J Biomed Mater Res Part B: Appl Biomater, 82B:156-168 (2007); and Miyazawa et al., Journal of Gastroenterology and Hepatology, 22:1959-1964 (2007)).

Cells may also be grown in “3D” (aggregated) cultures. An example is PCT/US2009/31528, filed Jan. 21, 2009.

Once established in culture, cells can be used fresh or frozen and stored as frozen stocks, using, for example, DMEM with 40% FCS and 10% DMSO. Other methods for preparing frozen stocks for cultured cells are also available to those of skill in the art.

### Pharmaceutical Formulations

U.S. Pat. No. 7,015,037 is incorporated by reference for teaching pharmaceutical formulations. In certain embodiments, the cell populations are present within a composition adapted for and suitable for delivery, i.e., physiologically compatible.

In some embodiments the purity of the cells (or conditioned medium) for administration to a subject is about 100% (substantially homogeneous). In other embodiments it is 95% to 100%. In some embodiments it is 85% to 95%. Particularly, in the case of admixtures with other cells, the percentage can be about 10%-15%, 15%-20%, 20%-25%, 25%-30%, 30%-35%, 35%-40%, 40%-45%, 45%-50%, 60%-70%, 70%-80%, 80%-90%, or 90%-95%. Or isolation/purity can be expressed in terms of cell doublings where the cells have undergone, for example, 10-20, 20-30, 30-40, 40-50 or more cell doublings.

The choice of formulation for administering the cells for a given application will depend on a variety of factors. Prominent among these will be the species of subject, the nature of the condition being treated, its state and distribution in the subject, the nature of other therapies and agents that are being administered, the optimum route for administration, survivability via the route, the dosing regimen, and other factors that will be apparent to those skilled in the art. For instance, the choice of suitable carriers and other additives will depend on the exact route of administration and the nature of the particular dosage form.

Final formulations of the aqueous suspension of cells/medium will typically involve adjusting the ionic strength of the suspension to isotonicity (i.e., about 0.1 to 0.2) and to physiological pH (i.e., about pH 6.8 to 7.5). The final formulation will also typically contain a fluid lubricant.

In some embodiments, cells/medium are formulated in a unit dosage injectable form, such as a solution, suspension, or emulsion. Pharmaceutical formulations suitable for injection of cells/medium typically are sterile aqueous solutions and dispersions. Carriers for injectable formulations can be a solvent or dispersing medium containing, for example, water, saline, phosphate buffered saline, polyol (for example, glycerol, propylene glycol, liquid polyethylene glycol, and the like), and suitable mixtures thereof.

The skilled artisan can readily determine the amount of cells and optional additives, vehicles, and/or carrier in compositions to be administered in methods of the invention. Typically, any additives (in addition to the cells) are present in an amount of 0.001 to 50 wt % in solution, such as in phosphate buffered saline. The active ingredient is present in the order of micrograms to milligrams, such as about 0.0001 to about 5 wt %, preferably about 0.0001 to about 1 wt %, most preferably about 0.0001 to about 0.05 wt % or about 0.001 to about 20 wt %, preferably about 0.01 to about 10 wt %, and most preferably about 0.05 to about 5 wt %.

In some embodiments cells are encapsulated for administration, particularly where encapsulation enhances the effectiveness of the therapy, or provides advantages in handling and/or shelf life. Cells may be encapsulated by membranes, as well as capsules, prior to implantation. It is contemplated that any of the many methods of cell encapsulation available may be employed.

A wide variety of materials may be used in various embodiments for microencapsulation of cells. Such materials include, for example, polymer capsules, alginate-poly-L-lysine-alginate microcapsules, barium poly-L-lysine alginate capsules, barium alginate capsules, polyacrylonitrile/polyvinylchloride (PAN/PVC) hollow fibers, and polyethersulfone (PES) hollow fibers.

Techniques for microencapsulation of cells that may be used for administration of cells are known to those of skill in the art and are described, for example, in Chang, P., et al., 1999; Matthew, H. W., et al., 1991; Yanagi, K., et al., 1989; Cai Z. H., et al., 1988; Chang, T. M., 1992 and in U.S. Pat. No. 5,639,275 (which, for example, describes a biocompatible capsule for long-term maintenance of cells that stably express biologically active molecules. Additional methods of encapsulation are in European Patent Publication No. 301,777 and U.S. Pat. Nos. 4,353,888; 4,744,933; 4,749,620; 4,814,274; 5,084,350; 5,089,272; 5,578,442; 5,639,275; and 5,676,943. All of the foregoing are incorporated herein by reference in parts pertinent to encapsulation of cells.

Certain embodiments incorporate cells into a polymer, such as a biopolymer or synthetic polymer. Examples of biopolymers include, but are not limited to, fibronectin, fibrin, fibrinogen, thrombin, collagen, and proteoglycans. Other factors, such as the cytokines discussed above, can also be incorporated into the polymer. In other embodiments of the invention, cells may be incorporated in the interstices of a three-dimensional gel. A large polymer or gel, typically, will be surgically implanted. A polymer or gel that can be formulated in small enough particles or fibers can be administered by other common, more convenient, non-surgical routes.

The dosage of the cells will vary within wide limits and will be fitted to the individual requirements in each particular case. In general, in the case of parenteral administration, it is customary to administer from about 0.01 to about 20 million cells/kg of recipient body weight. The number of cells will vary depending on the weight and condition of the recipient, the number or frequency of administrations, and other variables known to those of skill in the art. The cells can be administered by a route that is suitable for the tissue or organ. For example, they can be administered systemically, i.e., parenterally, by intravenous administration, or can be targeted to a particular tissue or organ; they can be administrated via subcutaneous administration or by administration into specific desired tissues.

The cells can be suspended in an appropriate excipient in a concentration from about 0.01 to about 5×10⁶ cells/ml. Suitable excipients for injection solutions are those that are biologically and physiologically compatible with the cells and with the recipient, such as buffered saline solution or other suitable excipients. The composition for administration can be formulated, produced, and stored according to standard methods complying with proper sterility and stability.

Administration into Lymphohematopoietic Tissues

Techniques for administration into these tissues are known in the art. For example, intra-bone marrow injections can involve injecting cells directly into the bone marrow cavity typically of the posterior iliac crest but may include other sites in the iliac crest, femur, tibia, humerus, or ulna; splenic injections could involve radiographic guided injections into the spleen or surgical exposure of the spleen via laparoscopic or laparotomy; Peyer's patches, GALT, or BALT injections could require laparotomy or laparoscopic injection procedures.

### Dosing

Doses for humans or other mammals can be determined without undue experimentation by the skilled artisan, from this disclosure, the documents cited herein, and the knowledge in the art. The dose of cells/medium appropriate to be used in accordance with various embodiments of the invention will depend on numerous factors. The parameters that will determine optimal doses to be administered for primary and adjunctive therapy generally will include some or all of the following: the disease being treated and its stage; the species of the subject, their health, gender, age, weight, and metabolic rate; the subject's immunocompetence; other therapies being administered; and expected potential complications from the subject's history or genotype. The parameters may also include: whether the cells are syngeneic, autologous, allogeneic, or xenogeneic; their potency (specific activity); the site and/or distribution that must be targeted for the cells/medium to be effective; and such characteristics of the site such as accessibility to cells/medium and/or engraftment of cells. Additional parameters include co-administration with other factors (such as growth factors and cytokines). The optimal dose in a given situation also will take into consideration the way in which the cells/medium are formulated, the way they are administered, and the degree to which the cells/medium will be localized at the target sites following administration.

The optimal dose of cells could be in the range of doses used for autologous, mononuclear bone marrow transplantation. For fairly pure preparations of cells, optimal doses in various embodiments will range from 10⁴ to 10⁸ cells/kg of recipient mass per administration. In some embodiments the optimal dose per administration will be between 10⁵ to 10⁷ cells/kg. In many embodiments the optimal dose per administration will be 5×10⁵ to 5×10⁶ cells/kg. By way of reference, higher doses in the foregoing are analogous to the doses of nucleated cells used in autologous mononuclear bone marrow transplantation. Some of the lower doses are analogous to the number of CD34⁺ cells/kg used in autologous mononuclear bone marrow transplantation.

In various embodiments, cells/medium may be administered in an initial dose, and thereafter maintained by further administration. Cells/medium may be administered by one method initially, and thereafter administered by the same method or one or more different methods. The levels can be maintained by the ongoing administration of the cells/medium. Various embodiments administer the cells/medium either initially or to maintain their level in the subject or both by intravenous injection. In a variety of embodiments, other forms of administration, are used, dependent upon the patient's condition and other factors, discussed elsewhere herein.

Cells/medium may be administered in many frequencies over a wide range of times. Generally lengths of treatment will be proportional to the length of the disease process, the effectiveness of the therapies being applied, and the condition and response of the subject being treated.

### Uses

Administering the cells is useful to reduce undesirable inflammation in any number of CNS pathologies, including, but not limited to, ischemic stroke, multiple sclerosis, Alzheimer's Disease, ALS, Parkinson's Disease, hypoxic-ischemia, neonatal hypoxic ischemia, and traumatic brain or spinal cord injury.

In addition, other uses are provided by knowledge of the biological mechanisms described in this application. One of these includes drug discovery. This aspect involves screening one or more compounds for the ability to affect the cell's ability to modulate microglia activation. This would involve an assay for the cell's ability to modulate the activation. Accordingly, the assay may be designed to be conducted in vivo or in vitro. Modulation assays could assess the activation state at any desired level, e.g., morphological, gene expression, functional, etc.

Cells (or medium) can be selected by the ability to modulate or by the expression of one or more genes that act as modulators. Gene expression can be assessed by directly assaying protein or RNA. This can be done through any of the well-known techniques available in the art, such as by FACS and other antibody-based detection methods and PCR and other hybridization-based detection methods. Indirect assays may also be used for expression, such as binding to any of the known receptors

Assays for potency may be performed by detecting the genes modulated by the cells. These may include, but are not limited to, oxygen radicals, NO, TNFα, Glu, quinolic acid, histamine, eicosanoids, NGF, BDNF, NT-4/5, TGFβ, GDNF, CNTF, IL-6, LIF, bFGF, HGF, PGn, IL-3, MMP-9, iNOS, CD16, CD86, CD64, and CD32, scavenger receptor A, CD163, arginase 1, CD14, CD206, CD23, and scavenger receptor B. Detection may be direct, e.g., via RNA or protein assays or indirect, e.g., biological assays for one or more biological effects of these genes. Alternatively, potency may be assayed by detecting the modulators of these genes, e.g., MMP modulators.

Accordingly, a surrogate marker could be used as long as it serves as an indicator that the cells express/secrete the factor(s) that modulate microglia activation.

Assays for expression/secretion of modulatory factors include, but are not limited to, ELISA, Luminex qRT-PCR, anti-factor western blots, and factor immunohistochemistry on tissue samples or cells.

Quantitative determination of modulatory factors in cells and conditioned media can be performed using commercially available assay kits (e.g., R&D Systems that relies on a two-step subtractive antibody-based assay).

In vitro activation assays can also be used to assess the expression/secretion of the factors. Such in vitro assays are well known in the art, for example, ELISA assay of secreted cytokines and growth factors, qRT-PCR analysis of genes associated with neurotoxic or neuroprotective activation state, and immunohistochemistry analysis of activation state marker expression.

A further use for the invention is the establishment of cell banks to provide cells for clinical administration. Generally, a fundamental part of this procedure is to provide cells that have a desired potency for administration in various therapeutic clinical settings.

Any of the same assays useful for drug discovery could also be applied to selecting cells for the bank as well as from the bank for administration.

Accordingly, in a banking procedure, the cells (or medium) would be assayed for the ability to achieve any of the effects disclosed herein (i.e., modulate microglia activation and the effects thereof, such as reduce inflammation, etc.). Then, cells would be selected that have a desired potency for any of the effects, and these cells would form the basis for creating a cell bank.

It is also contemplated that potency can be increased by treatment with an exogenous compound, such as a compound discovered through screening the cells with large combinatorial libraries. These compound libraries may be libraries of agents that include, but are not limited to, small organic molecules, antisense nucleic acids, siRNA DNA aptamers, peptides, antibodies, non-antibody proteins, cytokines, chemokines, and chemo-attractants. For example, cells may be exposed such agents at any time during the growth and manufacturing procedure. The only requirement is that there be sufficient numbers for the desired assay to be conducted to assess whether or not the agent increases potency. Such an agent, found during the general drug discovery process described above, could more advantageously be applied during the last passage prior to banking.

One embodiment that has been applied successfully to MultiStem is as follows. Cells can be isolated from a qualified marrow donor that has undergone specific testing requirements to determine that a cell product that is obtained from this donor would be safe to be used in a clinical setting. The mononuclear cells are isolated using either a manual or automated procedure. These mononuclear cells are placed in culture allowing the cells to adhere to the treated surface of a cell culture vessel. The MultiStem cells are allowed to expand on the treated surface with media changes occurring on day 2 and day 4. On day 6, the cells are removed from the treated substrate by either mechanical or enzymatic means and replated onto another treated surface of a cell culture vessel. On days 8 and 10, the cells are removed from the treated surface as before and replated. On day 13, the cells are removed from the treated surface, washed and combined with a cryoprotectant material and frozen, ultimately, in liquid nitrogen. After the cells have been frozen for at least one week, an aliquot of the cells is removed and tested for potency, identity, sterility and other tests to determine the usefulness of the cell bank. These cells in this bank can then be used by thawing them, placing them in culture or use them out of the freeze to treat potential indications.

Another use is a diagnostic assay for efficiency and beneficial clinical effect following administration of the cells. Depending on the indication, there may be biomarkers available to assess. These are, for example, downregulation of pro-inflammatory cytokines (TNF-α, IL-6, IL-1β, INF-γ) and upregulation of anti-inflammatory cytokines (IL-4, IL-10, TGF-β). The dosage of cells can be adjusted during treatment according to the effect.

A further use is to assess the efficacy of the cell to achieve any of the above results as a pre-treatment diagnostic that precedes administering the cells to a subject. Moreover, dosage can depend upon the potency of the cells that are being administered. Accordingly, a pre-treatment diagnostic assay for potency can be useful to determine the dose of the cells initially administered to the patient and, possibly, further administered during treatment based on the real-time assessment of clinical effect.

It is also to be understood that the cells of the invention can be used to modulate microglia activation not only for purposes of treatment, but also research purposes, both in vivo and in vitro to understand the mechanism involved in microglia activation, normally and in disease models. In one embodiment, assays, in vivo or in vitro, can be done in the presence of desired agents. The effect of those agents can then be assessed. These types of assay could also be used to screen for agents that have an effect on the activation that is modulated by the cells of the invention. Accordingly, in one embodiment, one could screen for agents in the disease model that reverse the negative effects and/or promote positive effects. Conversely, one could screen for agents that have negative effects in a model of normal activation.

### Compositions

The invention is also directed to cell populations with specific potencies for achieving any of the effects described herein. As described above, these populations are established by selecting for cells that have desired potency. These populations are used to make other compositions, for example, a cell bank comprising populations with specific desired potencies and pharmaceutical compositions containing a cell population with a specific desired potency.

### NON-LIMITING EXAMPLES

### Example 1

Hypothesis: Immunomodulatory effect of MultiStem® can moderate the activation profile of microglia.

### Rationale/Background

Immunomodulation within the CNS by microglia plays a significant role in defining disease/injury severity in a number of different CNS paradigms, including ischemic stroke, multiple sclerosis, Alzheimer's disease, and traumatic brain or spinal cord injury, among others (Hanisch & Kettenmann, 2007; Block et al., 2007). Within the context of such diseases or injuries, microglia become “activated,” leading to morphological changes, increased proliferation, and/or upregulation of a number of proteins of varied function. Activation by stressful stimuli can lead to either neuroprotection, via microglial secretion of neurotrophic and neuroprotective factors; or neurotoxicity, via secretion of a range of neurotoxic factors, such as the proinflammatory cytokines TNF-α, IL-6, and IL-1β. These two very different outcomes depend on the level and type of activation, and whether they are driven more toward a TH2 (neuroprotective) immune response, or a TH1 (neurotoxic) immune response (Biber et al., 2007).

Massive injuries or severe disease, such as ischemic stroke or multiple sclerosis, have been shown to induce activation toward a TH1 response (Hanisch & Kettenmann, 2007), which leads to increased neuronal death and toxicity through increased secretion of TNF-α, IL-6, matrix metalloproteinases, and other neurotoxic compounds. Neurotoxicity resulting from activated microglia is thought to constitute a significant component of the neuronal death associated with such injuries and diseases (Block et al., 2007). However, data also indicate that microglial activation does not necessarily result in neurotoxicity, and in fact can, in some cases, lead to enhanced neuroprotection (Biber et al., 2007; Kigerl et al., 2009). A number of molecules have been identified as specific regulators of activation profile, including factors that induce the neuroprotective activation state, such as CCL21 and CXCL10; and factors that suppress the neurotoxic activation state, such as TGF-β, CCL5, and NGF (Biber et al., 2007; Gamo et al., 2008).

The inventors have found that a number of these factors are expressed by MultiStem®. Additional studies have demonstrated that MultiStem® displays potent immunosuppressive properties toward other immune cell types. Modulation of microglial activation presents an important potential mechanism through which MultiStem® may provide significant clinical benefit in a number of central nervous system indications. Within this study, the inventors examined whether MultiStem® could alter the microglial activation profile in response to lipopolysaccharide (LPS), a potent activator of the neurotoxic activation state of microglia. The inventors found that co-culture of human MultiStem® with BV2 mouse microglial cells significantly inhibits release of microglial TNF-α, IL-6, and MMP-9 upon stimulation with LPS. This effect does not appear to be due to direct inhibition of signal detection or uptake of the above-mentioned factors, as similar inhibition is achieved via pre-incubation of the microglial cells with MultiStem® prior to LPS stimulation. Finally, the suppression of release of these proinflammatory molecules also does not appear to require signaling to MultiStem®, as treating microglia with conditioned media from cultured MultiStem® is also capable of inhibiting microglial secretion of TNF-α, IL-6, and MMP-9.

Microglial stimulation by LPS in the presence of MultiStem® co-culture prevents microglial upregulation of pro-inflammatory molecules. BV2 mouse microglial cells were plated at 1×10⁵ cells/ml, in the presence of human MultiStem® added at 2×10⁵ cells/ml via TransWells. Control wells received TransWells with no MultiStem® added. Co-cultures were maintained for 24 hours, followed by 24 hours stimulation with LPS (5 μg/ml). Media was collected from the co-cultures and analyzed by ELISAs specific for mouse TNF-α (A), IL-6 (B), and MMP-9 (C) (R&D Systems, Inc.).

### Results

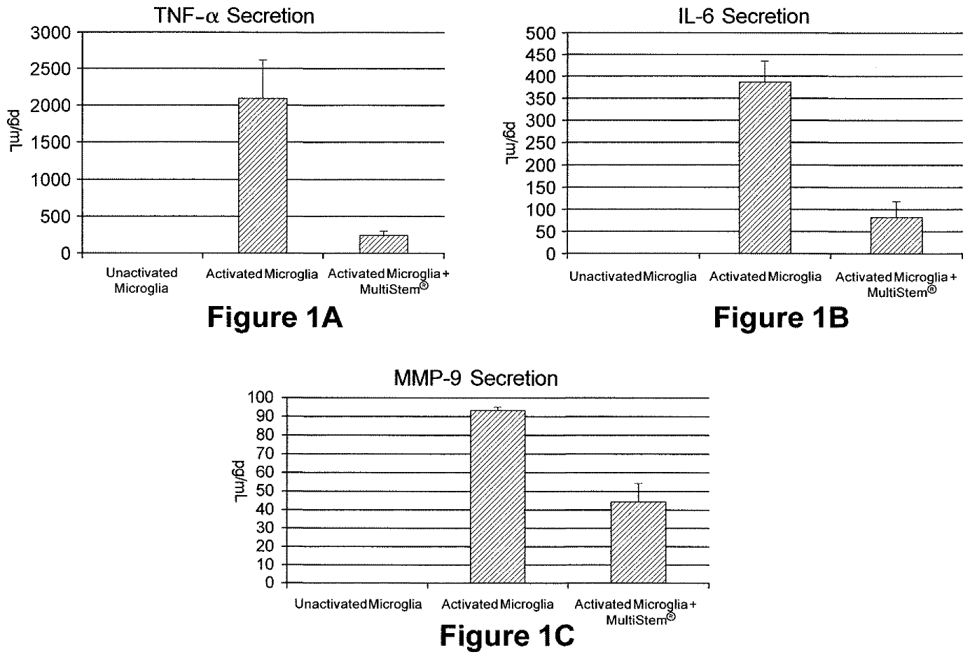

Co-Culture of human MultiStem® with BV2 mouse microglial cells inhibits the release of proinflammatory molecules from microglia upon activation with LPS. Activation of microglia with noxious stimuli such as LPS causes rapid and robust release of a number of proinflammatory cytokines, such as TNF-α and IL-6, and matrix metalloproteinases, such as MMP-9. To test whether MultiStem® can modulate this effect, human MultiStem® was co-cultured via TransWells with BV2 mouse microglial cells for 24 hours. LPS (5 μg/ml) was added to the cultures to induce microglial activation, and the media was collected 24 hours later. Mouse TNF-α, IL-6, and MMP-9 within the media were measured by ELISA (FIG. 1). All three molecules were undetectable or nearly undetectable in nonactivated microglial media, as well as in media from nonactivated microglia co-cultured with MultiStem®. Activation of microglia by themselves resulted in significant secretion of TNF-α, IL-6, and MMP-9 24 hours after LPS activation. However, LPS activation of microglia in the presence of human MultiStem® resulted in significant inhibition of microglial secretion of TNF-α (FIG. 1A), IL-6 (FIG. 1B), and MMP-9 (FIG. 1C). TNF-α, IL-6, and MMP-9 levels were decreased 89%, 79%, and 54%, respectively.

Pre-incubation of microglial cells with human MultiStem® prior to LPS stimulation results in lasting inhibition of proinflammatory secretion. The decreased presence of TNF-α, IL-6, and MMP-9 detected in the media of LPS-treated microglia co-cultured with MultiStem® could be due to a number of different mechanisms, such as decreased release, uptake by MultiStem®, or binding by molecules released from MultiStem®. To verify that the decreased signal detected by ELISA is due to decreased release of the measured factors from the microglial cells, a pre-incubation co-culture paradigm was performed. BV2 microglial cells were co-cultured with human MultiStem® via TransWells for 24 hours. MultiStem® was then removed from the microglial cells and discarded. Control wells were treated identically, but with TransWells containing no MultiStem®. The microglial cultures were washed three times with PBS, and were then fed with fresh media. After three hours, the microglial cultures were stimulated with LPS (5 μg/ml×24 hours), and the media was collected for analysis.

Microglia secretion of TNF-α and IL-6 after pre-incubation with human MultiStem® showed similar inhibition (FIG. 2A,B) as seen in direct co-culture experiments (FIG. 1A,B). TNF-α was decreased by 94% in MultiStem®-pre-incubated cultures (FIG. 2A); and IL-6 was decreased by 71% in MultiStem®-pre-incubated cultures (FIG. 2B). MMP-9 secretion was not inhibited by pre-incubation of microglial cells with MultiStem® (FIG. 2C).

Pre-incubation of microglial cells with MultiStem® results in prolonged inhibition of microglial activation potential as measured by release of proinflammatory molecules after stimulation with LPS. BV2 mouse microglial cells were plated at 1×10⁵ cells/ml, in the presence of human MultiStem® added at 2×10⁵ cells/ml via TransWells. Control wells received TransWells with no MultiStem® added. Co-cultures were maintained for 24 hours, after which the TransWells (containing MultiStem®) were discarded. Microglia were washed 3× with PBS, and fed with fresh media. After 3 hours, microglia were stimulated with LPS (5 μg/ml). After 24 hours LPS stimulation, media was collected from the microglia and analyzed by ELISAs specific for mouse TNF-α (FIG. 2A), IL-6 (FIG. 2B), and MMP-9 (FIG. 2C).

Microglial secretion of pro-inflammatory molecules is inhibited by MultiStem® conditioned media. To determine whether MultiStem's effects on microglial activation require signaling to MultiStem®, experiments were performed treating microglia with MultiStem® conditioned media, rather than direct co-culture or co-culture pre-incubation. 4-day conditioned media was collected from human MultiStem® cultures grown in isolation (not exposed to microglia). Microglial cultures had their media removed, and replaced with the conditioned media harvested from MultiStem®. Control microglial cultures received MultiStem® basal media. After 3 hours incubation in the MultiStem® basal or conditioned medias, the microglia were stimulated with LPS (5 μg/ml×24 hours), and the media was collected for analysis.

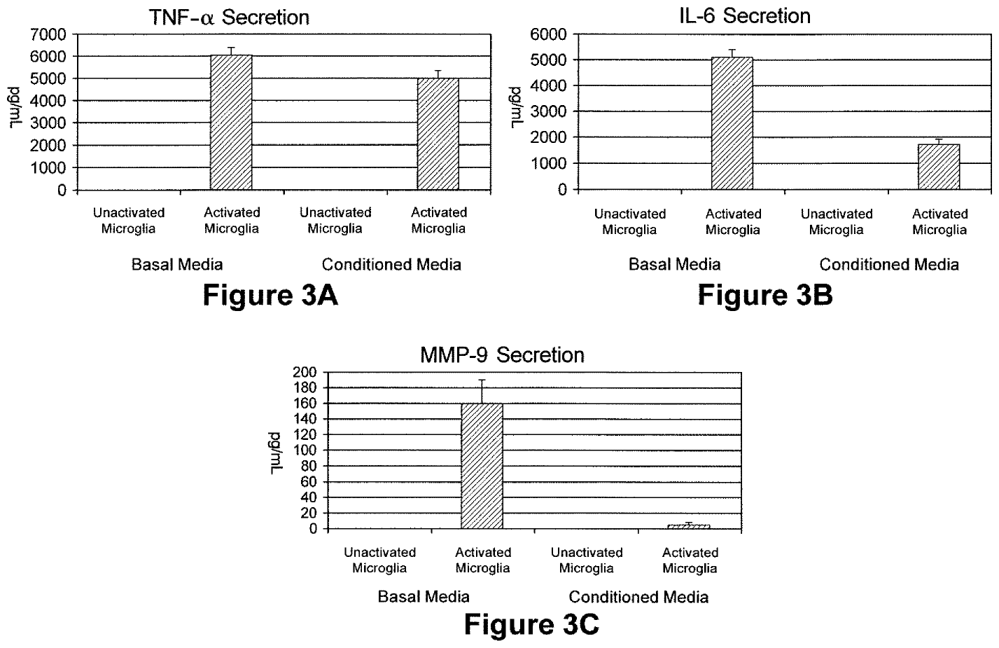

TNF-α secretion was undetectable when unactivated microglia were cultured in MultiStem® basal or conditioned media. Stimulation with LPS induced robust TNF-α secretion by microglia in the basal media. LPS stimulation within MultiStem® conditioned media resulted in slightly decreased secretion of TNF-α (19% decrease; FIG. 3A).

Treatment of microglial cells with conditioned media from MultiStem® inhibits microglial release of proinflammatory molecules in response to LPS stimulation. BV2 mouse microglial cells were plated at 1×10⁵ cells/ml. The media on the microglial cells was replaced with either MultiStem® basal media (controls), or conditioned media collected from nascent human MultiStem® cultures. After 3 hours, the microglia were stimulated with LPS (5 μg/ml), in the presence of the basal or conditioned medias. After 24 hours LPS stimulation, media was collected from the microglial cells and analyzed by ELISAs specific for mouse TNF-α (FIG. 3A), IL-6 (FIG. 3B), and MMP-9 (FIG. 3C).

IL-6 was similarly undetectable in media from unactivated microglia, whether they were cultured in basal or conditioned MultiStem® media. LPS stimulation in basal media resulted in a robust increase in IL-6 secretion, which was significantly inhibited (66% reduction) in the presence of MultiStem® conditioned media (FIG. 3B).

MMP-9 also was undetectable in media from unactivated microglia (in both basal and conditioned medias). MMP9 secretion was readily detectable after LPS activation in basal media, but was almost completely inhibited (97% reduction) after LPS activation in conditioned media (FIG. 3C).

### CONCLUSION

In the present study, the inventors investigated whether human MultiStem® could modulate the activation profile of mouse microglial immune cells in response to LPS, a potent activator that typically results in significant secretion of highly neurotoxic molecules. The results indicate that MultiStem® dramatically alters the response of microglial cells to LPS stimulation. In the direct presence of human MultiStem® (via TransWell co-culture), LPS-induced microglial secretion of the prototypical neurotoxic proinflammatory molecule, TNF-α, is almost completely inhibited (89% reduction; FIG. 1A). Release of IL-6, another neurotoxic proinflammatory cytokine, is inhibited almost as efficiently (79% reduction;

FIG. 1B). Both molecules are highly toxic to neurons, and have been shown to be released from microglia, and responsible for significant neuronal death, in a number of CNS disease and injury models (McCoy & Tansey, 2008). Interestingly, MultiStem® has been shown previously to shed significant levels of soluble TNF receptor I (sTNFRI), which is capable of binding and inhibiting secreted TNF-α. The shedding of sTNFRI by MultiStem® within the direct co-culture paradigm could be partially responsible for the decreased TNF-α signal detected by ELISA in FIG. 1A. However, in the MultiStem® pre-incubation paradigm, the decrease in detectable TNF-α is almost identical (94% reduction; FIG. 2A) to that seen in the direct presence of MultiStem®. In this paradigm, MultiStem® has been removed from the microglial cultures, and the microglia have been washed repeatedly and fed with fresh media, which would remove any MultiStee-shed sTNFRI. IL-6 detection is similarly decreased (71% reduction; FIG. 2B) after MultiStem® pre-incubation. These data demonstrate that human MultiStem® is capable of inhibiting microglial release of neurotoxic TNF-α and IL-6 after stimulation with LPS. Interestingly, LPS stimulation of microglia in the presence of MultiStem® conditioned media results in similar inhibition of IL-6 release (66% reduction; FIG. 3B), but only a slight decrease in TNF-α release (19% reduction; FIG. 3A). This difference suggests different mechanisms of action, which are currently being investigated in ongoing experiments.

Secretion of the matrix metalloproteinase MMP-9 is also significantly repressed in the presence of MultiStem® (54% reduction; FIG. 1C). MMP-9 secretion is thought to contribute to extracellular matrix digestion, myelin degradation, axonal damage, and neuronal death in a number of neurodegenerative diseases (Rosenberg, 2009; Yong et al., 2007). Importantly, the measurement of MMP-9 in the present experiments was performed using an ELISA that recognizes all forms of MMP-9 (active MMP-9, pro-MMP-9, and TIMP-MMP-9 complexes). The significant decrease in total MMP-9 detected both in the presence of human MultiStem®, as well as in the presence of MultiStem® conditioned media (97% reduction; FIG. 3C), indicates that MultiStem® inhibits microglial release of MMP-9, regardless of whether it is the active form, the pro- form, or bound in a complex by the MMP-inhibiting TIMPs. Interestingly, after pre-incubation with MultiStem® followed by LPS stimulation, no inhibition of MMP-9 secretion was observed (FIG. 2C), even though TNF-α and IL-6 secretion were significantly reduced. Because the MMP-9 ELISA used in these experiments detects all forms of MMP-9, it is unlikely that the observed decreases in MMP-9 signal in the direct presence of MultiStem® and in the presence of MultiStem® conditioned media are due to inhibition of pro-MMP-9 cleavage, or binding of MMP-9 by TIMPs. It is also not due to uptake of MMP-9 by MultiStem®, as MultiStem® cells are not present within the conditioned media paradigm. Rather, the reduced MMP-9 signal measured in the presence of MultiStem® co-culture or MultiStem® conditioned media is most likely due to repression of microglial secretion. The lack of inhibition of MMP-9 secretion after MultiStem® pre-incubation may be due to cross-talk between the two cell types during the pre-incubation portion of the paradigm. Importantly, during the pre-incubation phase, the microglial cells have not yet been stimulated with LPS, and the MultiStem® cells are only being exposed to normal, unactivated microglia. Activation of the microglial cells prior to pre-incubation with MultiStem®, such that MultiStem® is exposed to activated microglia, may produce different results. Experiments to investigate this possibility and the responsible mechanism are ongoing.

The importance of microglial chronic activation toward a TH1 immune phenotype and the resulting neurotoxicity is well documented within a range of CNS injuries and neurodegenerative disorders (Block et al., 2007). Microglial release of TNF-α, IL-6, and MMP-9 is considered as a prototypical indicator of activation toward a highly neurotoxic TH1 immune phenotype. The impressive inhibition of microglial secretion of these molecules after LPS stimulation, not only in the direct presence of human MultiStem® but also in the presence of MultiStem® conditioned media and after pre-incubation with MultiStem®, indicates a profound ability of MultiStem® to immunomodulate the response of microglial cells to a typical noxious stimulus. Secretion of additional proinflammatory cytokines, as well as neurotoxic molecules such as nitric oxide, and hydrogen peroxide, are commonly associated with microglial secretion of TNF-α, IL-6, and MMP-9. Secretion of these toxic molecules may be similarly inhibited by MultiStem®, and ongoing experiments are investigating this possibility. Additionally, when shifted away from the neurotoxic TH1 activation profile, and toward the neuroprotective TH2 activation profile, microglia not only downregulate secretion of proinflammatory and other neurotoxic molecules, but also typically upregulate secretion of neuroprotective growth factors. Ongoing experiments are testing whether modulation of microglial activation by MultiStem® may increase the microglial secretion of such neuroprotective factors.

### REFERENCES

Biber, K. et al. “Neuronal ‘On’ and ‘Off’ signals control microglia” Trends Neurosci 30(11):596-602 (2007).
Block, M. L. et al., “Microglia-mediated neurotoxicity: uncovering the molecular mechanisms” Nat Rev Neurosci 8:57-69 (2007).
Gamo, K. et al., “G-protein-coupled receptor screen reveals a role for chemokine receptor CCR5 in suppressing microglial neurotoxicity” J Neurosci 28(46):11980-11988 (2008).
Hanisch, U.-K., and Kettenmann, H., “Microglia: active sensor and versatile effector cells in the normal and pathologic brain” Nat Neurosci 10(11):1387-1394(2007).
Kigerl, K. et al., “Identification of two distinct macrophage subsets with divergent effects causing either neurotoxicity or regeneration in the injured mouse spinal cord” J Neurosci 29(43):13435-13444.
McCoy, M. K., and Tansey, M. G., “TNF signaling inhibition in the CNS: implications for normal brain function and neurodegenerative disease” J Neuroinflammation 5:45-58 (2008).
Rosenberg, G. A. “Matrix metalloproteinases and their multiple roles in neurodegenerative diseases” Lancet Neurol 8:205-216 (2009).
Yong, V. W. et al., “Targeting MMPs in acute and chronic neurological conditions” Neurotherapeutics 4:580-589 (2007).

### Example 2

### Effects of MultiStem® on Ischemia-Induced Gene Expression Changes

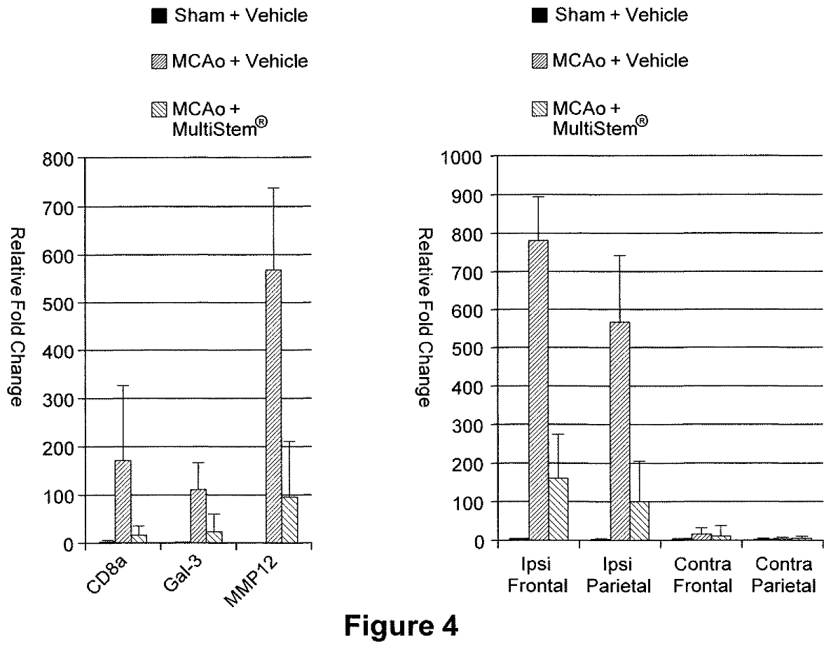

MultiStem significantly inhibits ischemia-induced markers of immune response with infarct region (FIG. 4). qPCR analysis confirms that MultiStem administration prevents massive ischemia-induced upregulation of markers of immune response, such as CD8a, Gal-3, and MMP12 (expressed by T cells, and microglia). qPCR analysis of different brain regions demonstrates that the changes in immune marker genes are specific to the infarct region.

Neonatal Hypoxia-Ischemia Paradigm. Neonatal hypoxia-ischemia (HI) was administered to neonatal rats on postnatal day 7 (P7). P7 pups underwent permanent ligation of the CCA, followed by exposure to 8% 02, 92% N2 for 2.5 hours. Animals were administered MultiStem® (100,000 or 1 million cells) or vehicle IV 7 days after injury. Animals were sacrificed 3 days after cell infusion for tissue harvest.

qPCR Analysis. First-strand cDNA was prepared from 500 ng RNA using MMLV RT under standard conditions. SYBR Green qPCR was performed on an Applied Biosystems 7500 PCR machine. Results were normalized to β-actin, and expressed as average percent of rat reference RNA (Stratagene).

1. A method for treating inflammation in a subject having a condition, the method comprising selecting cells that have a desired potency for modulating microglia activation, assaying the selected cells for the desired potency, and administering the cells having the desired potency to the subject, in a therapeutically effective amount and for a time sufficient to achieve a therapeutic result, the cells being non-embryonic stem, non-germ cells that express one or more of oct4, telomerase, rex-1, or rox-1 and/or can differentiate into cell types of at least two of endodermal, ectodermal, and mesodermal germ layers.

2. A method for treating inflammation in a subject having a condition, the method comprising administering cells having a desired potency for modulating microglia activation to the subject, in a therapeutically effective amount and for a time sufficient to achieve a therapeutic result, the cells being non-embryonic stem, non-germ cells that express one or more of oct4, telomerase, rex-1, or rox-1 and/or can differentiate into cell types of at least two of endodermal, ectodermal, and mesodermal germ layers, wherein, prior to administration, the cells were validated as having the desired potency.

3. A method for modulating microglia activation in a subject having a condition, the method comprising selecting cells that have a desired potency for modulating microglia activation, assaying the cells for the desired potency, and administering the cells having the desired potency to the subject, in a therapeutically effective amount and for a time sufficient to achieve a therapeutic result, the cells being non-embryonic stem, non-germ cells that express one or more of oct4, telomerase, rex-1, or rox-1 and/or can differentiate into cell types of at least two of endodermal, ectodermal, and mesodermal germ layers.

4-13. (canceled)
